(19) United States
(12) Patent Application Publication
(10) Pub. No.: US 2025/0177212 A1
(43) Pub. Date: Jun. 5, 2025

(54) HYDROCOLLOID-BASED SKIN PATCH

(71) Applicant: Slaydot LLC, Phoenix, AZ (US)

(72) Inventors: Kerim ERAVCI, Phoenix, AZ (US); Jeffrey Todd RUCH, Dana Point, CA (US)

(21) Appl. No.: 18/527,781

(22) Filed: Dec. 4, 2023

(51) Int. Cl. A61F 13/00; A61F 13/02; A61L 15/40

(52) U.S. Cl. CPC A61F 13/00063; A61F 13/0213; A61F 13/0259; A61F 13/0283; A61L 15/40; A61F 2013/0296

(57) ABSTRACT

A hydrocolloid-based skin patch that includes ozonated olive oil absorbed within a hydrocolloid layer, and a release layer overlying one side of the hydrocolloid layer.

### FIELD

This disclosure relates to systems and methods relating to cosmetic treatment of skin conditions, such as acne. More specifically, the disclosed embodiments relate to hydrocolloid-based cosmetic patches, and methods of manufacturing such patches.

### INTRODUCTION

Hydrocolloid materials are known to have absorbent and non-skin sensitizing characteristics, and are used in a variety of applications, including wound dressings, food additives, and drug delivery systems. Hydrocolloid dressings are used to treat mildly exuding wounds, such as minor burns or bed sores, by providing a moist and insulating environment that promotes wound healing. These dressings are waterproof and have an inner layer of gel-forming agents called hydrocolloid particles, which absorb exudate and provide a moist healing environment, allowing for faster healing and reduced scar formation. Hydrocolloid dressings are also easy to apply and remove, making them suitable for the pediatric population.

Hydrocolloid-based cosmetic patches are a type of hydrocolloid dressing that can be used to treat acne cosmetically. They are designed to be placed directly on top of a pimple and work by absorbing excess oil and pus, while also providing a moist environment that promotes healing. However, existing hydrocolloid-based cosmetic patches are known to have undesirable side effects in some cases, such as drying out or irritating the affected area. Accordingly, there is a need for new and improved hydrocolloid-based acne patches that can treat the affected area with a lesser chance of undesirable side effects.

### SUMMARY

The present disclosure provides systems, apparatuses, and methods relating to a hydrocolloid-based patch that includes ozonated olive oil absorbed within the hydrocolloid material.

In some embodiments, a hydrocolloid-based patch according to the present teachings may include an inner layer including a hydrocolloid material; ozonated olive oil absorbed within the hydrocolloid material of the inner layer; and a release layer overlying one side of the inner layer.

In some embodiments, a hydrocolloid-based patch according to the present teachings may further comprise an outer sealing layer overlying a side of the inner layer opposite the release layer.

In some embodiments, a hydrocolloid-based patch according to the present teachings may include a ratio of ozonated olive oil to hydrocolloid material in the inner layer is in the range of 0.0025 to 0.025 by weight; 0.005 to 0.010 by weight; 0.010 to 0.020 by weight; 0.007 to 0.008 by weight; or 0.0070 to 0.0075 by weight.

In some embodiments, a hydrocolloid-based patch according to the present teachings may include a hydrocolloid material that consists essentially of a pine-based hydrocolloid.

In some embodiments, a hydrocolloid-based patch according to the present teachings may include ozonated olive that is fully saturated with ozone, and/or ozonated olive oil having a density in the range of 0.800 g/cm³ to 1.25 g/cm³; having a density in the range of 0.930 g/cm³ to 1.10 g/cm³; or having a density in the range of 0.980 g/cm³ to 0.995 g/cm³.

In some embodiments, a method of manufacturing a skin patch for acne treatment may comprise ozonating olive oil until the olive oil is fully saturated with ozone; providing a planar expanse of material including a hydrocolloid material; coating a side of the planar expanse of material with the ozonated olive oil; applying a release layer to a side of the planar expanse of material; and cutting the planar expanse of acne treatment material of into patches of one or more sizes suitable for application to human skin.

In some embodiments, coating a side of the planar expanse of material with the ozonated olive oil includes coating exactly one side of the planar expanse of material with the ozonated olive oil. In some embodiments, the release layer is applied to the side of the planar expanse of material opposite the side of the planar expanse of material coated with ozonated olive oil.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is an exploded isometric view of an illustrative hydrocolloid-based acne treatment patch, in accordance with aspects of the present disclosure.

FIG. 2 is a schematic sectional view of a portion of the hydrocolloid-based acne treatment patch of FIG. 1, depicting ozonated olive oil absorbed within the hydrocolloid material.

FIG. 3 is a flow chart depicting steps of an illustrative method for manufacturing a hydrocolloid-based acne treatment patch, in accordance with aspects of the present disclosure.

### DETAILED DESCRIPTION

Various aspects and examples of a hydrocolloid-based acne treatment patch that includes ozonated olive oil absorbed within the hydrocolloid material, as well as related methods of manufacture, are described below and illustrated in the associated drawings. Unless otherwise specified, a hydrocolloid-based acne treatment patch in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

This Detailed Description includes the following sections, which follow immediately below: (1) Definitions; (2) Overview; (3) Examples, Components, and Alternatives; (4) Advantages, Features, and Benefits; and (5) Conclusion. The Examples, Components, and Alternatives section is further divided into subsections, each of which is labeled accordingly.

### Definitions

The following definitions apply herein, unless otherwise indicated.

“Comprising,” “including,” and “having” (and conjugations thereof) are used interchangeably to mean including but not necessarily limited to, and are open-ended terms not intended to exclude additional, unrecited elements or method steps.

Terms such as “first,” “second,” and “third” are used to distinguish or identify various members of a group, or the like, and are not intended to show serial or numerical limitation.

“AKA” means “also known as,” and may be used to indicate an alternative or corresponding term for a given element or elements.

“Colloid” means any mixture in which microscopically dispersed insoluble particles are suspended throughout another substance.

“Hydrocolloid” means any substance that forms a colloid when combined with water.

The terms “inner,” “outer,” and the like are intended to be understood in the context of a skin patch configured to be applied to human skin, where “inner” represents a location relatively closer to the skin, and “outer” represents a location relatively further from the skin.

The term “planar expanse” means locally formed or configured to be formed into the shape of an approximately flat, planar surface. For example, thin, flexible material that passes between rollers in a roll-to-roll processing system is a “planar expanse” of material because it is configured to be formed into a planar surface.

“Providing,” in the context of a method, may include receiving, obtaining, purchasing, manufacturing, generating, processing, preprocessing, and/or the like, such that the object or material provided is in a state and configuration for other steps to be carried out.

In this disclosure, one or more publications, patents, and/or patent applications may be incorporated by reference. However, such material is only incorporated to the extent that no conflict exists between the incorporated material and the statements and drawings set forth herein. In the event of any such conflict, including any conflict in terminology, the present disclosure is controlling.

### Overview

In general, a hydrocolloid-based acne treatment patch that includes ozonated olive oil absorbed within the hydrocolloid material may include a hydrocolloid layer comprising any type of hydrocolloid material suitable for application to human skin, ozonated olive oil absorbed within the hydrocolloid layer, and a release layer overlying one side of the hydrocolloid layer, to allow convenient storage and application of the treatment patch. In some cases, the ozonated olive oil may be fully saturated with ozone and/or may have a density that falls into a particular range. In some cases, the ratio of ozonated olive oil weight to hydrocolloid weight in the hydrocolloid layer falls into a particular range. In some cases, a sealing layer is provided overlying a side of the hydrocolloid layer opposite the release layer to prevent absorption of dirt and other contaminants into the hydrocolloid material.

### Examples, Components, and Alternatives

The following sections describe selected aspects of illustrative a hydrocolloid-based acne treatment patches that include ozonated olive oil absorbed within the hydrocolloid material, as well as related systems and/or methods. The examples in these sections are intended for illustration and should not be interpreted as limiting the scope of the present disclosure. Each section may include one or more distinct embodiments or examples, and/or contextual or related information, function, and/or structure.

### A. Illustrative Acne Treatment Patch

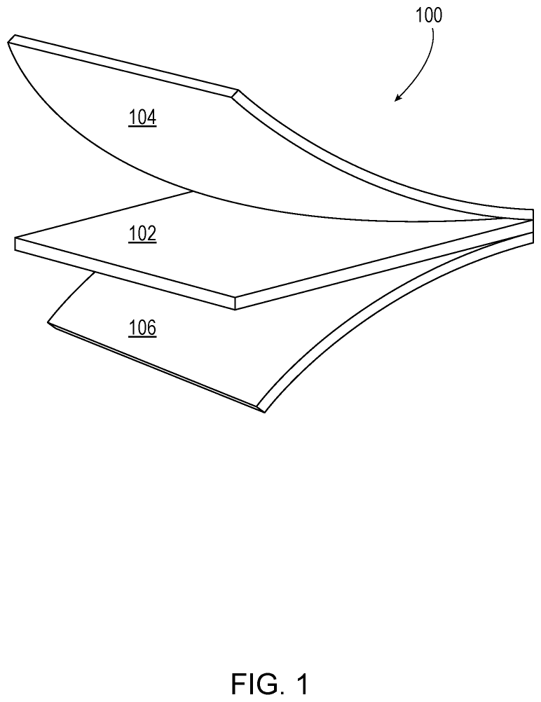

As shown in FIG. 1, this section describes an illustrative acne treatment patch that includes a hydrocolloid material layer, within which ozonated olive oil is absorbed. The illustrative treatment patch is an example of the general category of acne treatment patches that include a combination of hydrocolloid material and ozonated olive oil, according to aspects of the present teachings.

More specifically, FIG. 1 depicts a hydrocolloid-based patch, generally indicated at 100, which includes a hydrocolloid-based inner layer 102, an optional outer sealing layer 104 overlying one side of the inner layer, and an optional release layer 106 overlying a side of the inner layer opposite the outer sealing layer.

Inner layer 102 may include any suitable hydrocolloid, such as (for example) pectin, agar, carrageenan, gum Arabic, xanthan gum, guar gum, locust bean gum, methyl cellulose, sodium alginate, tara gum, and/or the like. In some cases, hydrocolloid material 108 may include a pine-based hydrocolloid. In some cases, inner layer 102 may include a hydrogel polymer. In some cases, inner layer 102 may include a combination of hydrocolloids and/or hydrogel polymers.

Outer sealing layer 104 may be any suitable layer configured to protect the inner layer 102 and the user's underlying skin from unwanted dirt, moisture, bacteria, and other contaminants. For instance, sealing layer 104 may be constructed from a non-woven fabric, a foam, a polyurethane film, and/or a combination of those or similar materials. In some cases, sealing layer 104 may be omitted as a separate layer, in which case an outer side of inner layer 102 may function as an outer sealing layer or protective layer.

Release layer 106 (which also may be referred to as a “release liner”) holds inner layer 102 and outer sealing layer 104 (if present) in place until a user is ready to apply patch 100 to the user's skin, at which point the user peels inner layer 102 away from the release layer, and applies the patch. Accordingly, release layer 106 should have properties configured, for example, to protect the inner layer from contamination, prevent the inner layer from drying out prematurely, and/or allow the inner layer to be peeled away from the release layer readily. Suitable materials for release layer 106 may include, for example, polyethylene terephthalate (PET), polyurethane, and silicone, among others. In some cases, release layer 106 may be perforated to allow convenient separation of discrete skin patches or subsets of patches prior to removal of the release layer.

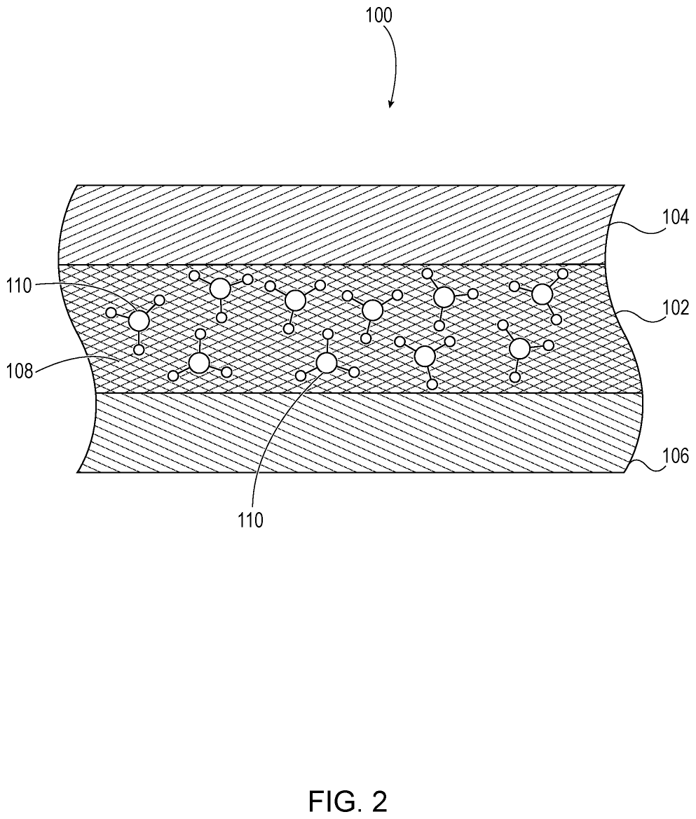

FIG. 2 is a schematic sectional view of a portion of the hydrocolloid-based patch of FIG. 1, showing more details of inner layer 102. Specifically, according to aspects of the present teachings, inner layer 102 includes a hydrocolloid material 108, and ozonated olive oil 110 absorbed within the hydrocolloid material.

Hydrocolloid material 108 may consist of or comprise any of the inner layer hydrocolloid materials discussed previously, such as pectin, agar, carrageenan, gum Arabic, xanthan gum, guar gum, locust bean gum, methyl cellulose, sodium alginate, tara gum, a pine-based hydrocolloid, a combination of hydrocolloid and/or hydrogel materials, and/or the like.

Ozonated olive oil 110 is olive oil within which ozone has been infused until it reacts with the oil to produce a relatively thicker oil or gel. The chemical structure has been studied to some extent, and is believed to include ozonides and/or peroxides, such as various isomers of triolein triozonide. In any event, ozonated olive oil is widely believed to have therapeutic properties, such as disinfecting and promoting healing of topical skin lesions.

Ozonated olive oil may be prepared by bubbling ozone-oxygen gas through pure olive oil for some desired period of time. For instance, in an exemplary process, an oxygen-ozone mixture may be bubbled through cold pressed, extra virgin olive oil for approximately 24 hours, resulting in ozonated olive oil which is fully saturated with ozone, meaning no additional ozone will be absorbed if the ozonation process is extended further in duration. Ozonated olive oil may be characterized by its density. According to the present teachings, for example, suitable densities of ozonated olive oil 110 for use in hydrocolloid-based patch 100 may be in the range of 0.930 g/cm³ to 1.10 g/cm³, or in the range of 0.980 g/cm³ to 0.995 g/cm³.

Inner layer 102 may be prepared by coating hydrocolloid material 108 with ozonated olive oil 110, on either one side or both sides of the hydrocolloid material, and allowing the ozonated olive oil to penetrate into and be absorbed by the hydrocolloid material. The resulting inner layer may be characterized by the ratio of ozonated olive oil 110 to hydrocolloid material 108 by weight. According to the present teachings, for example, suitable ratios of ozonated olive oil to hydrocolloid material may be in the range of 0.0025 to 0.025 by weight; in the range of 0.005 to 0.010 by weight; in the range of 0.010 to 0.020 by weight; or in the range of 0.007 to 0.008 by weight.

### B. Illustrative Method of Manufacturing

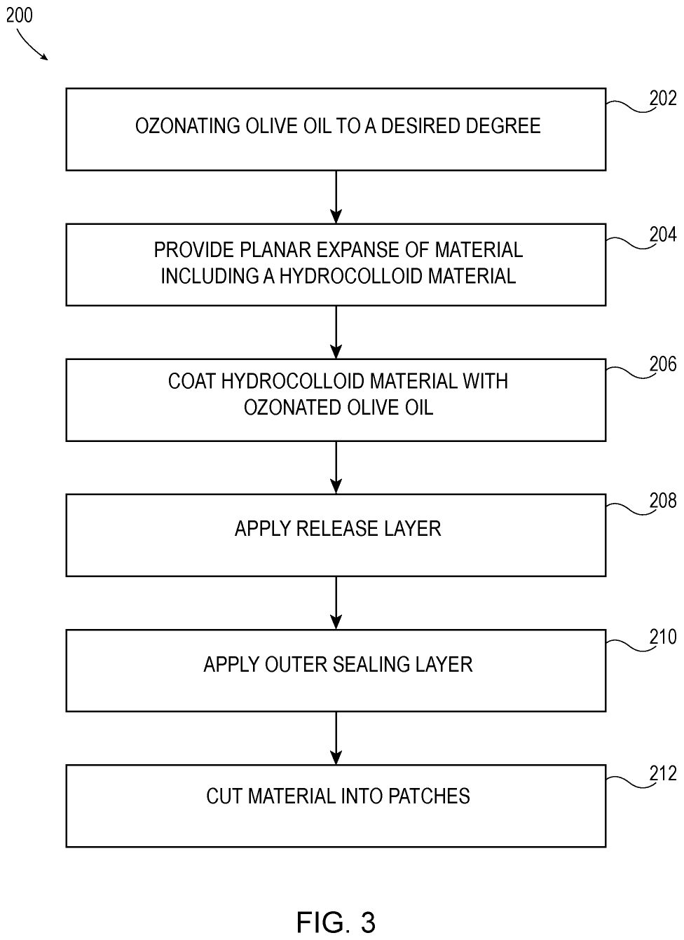

This section describes steps of an illustrative method 200 for manufacturing hydrocolloid-based patches that include a hydrocolloid material layer within which ozonated olive oil is absorbed in accordance with aspects of the present teachings; see FIG. 3. Aspects of the various layers comprised in illustrative hydrocolloid-based patch 100 described previously may be utilized in the method steps described below. Where appropriate, reference may be made to components and systems that may be used in carrying out each step. These references are for illustration, and are not intended to limit the possible ways of carrying out any particular step of the method.

FIG. 3 is a flowchart illustrating steps performed in an illustrative method, and may not recite the complete process or all steps of the method. Although various steps of method 200 are described below and depicted in FIG. 3, the steps need not necessarily all be performed, and in some cases may be performed simultaneously or in a different order than the order shown.

Step 202 of method 200 includes ozonating olive oil to a desired degree, such as until the olive oil is fully saturated with ozone or saturated with some lesser amount of ozone. As discussed previously, the ozonated olive oil resulting from step 202 may be characterized by its density, and suitable densities of ozonated olive oil for use in conjunction with the present teachings may be, for example, in the range of 0.930 g/cm³ to 1.10 g/cm³, or in the range of 0.980 g/cm³ to 0.995 g/cm³. Step 202 may be performed by any suitable method, such as bubbling an oxygen-ozone mixture through olive oil for some period of time. In an exemplary process, a mixture of approximately 90% oxygen and 10% ozone by weight may be combined with cold pressed extra virgin olive oil in a reactor at flow rates of two liters per minute of ozone and one liter per minute of olive oil (i.e., at a 2:1 flow ratio) for 24 hours, resulting in fully saturated ozonated olive oil.

Step 204 of method 200 includes providing a planar expanse of material including a hydrocolloid material. For instance, a hydrocolloid or hydrocolloid-containing material may be mixed and then extruded between rollers and/or through a narrow slot, to form a thin, flexible planar expanse of hydrocolloid material. In some cases, the planar expanse of hydrocolloid-containing material may be extruded onto a release layer, which may be either a temporary release layer used during the manufacturing process, or a more permanent release layer retained in the finished product until the user applies the product.

Suitable materials for use in preparing the planar expanse of hydrocolloid-containing material include the hydrocolloid materials discussed previously, such as pectin, agar, carrageenan, gum Arabic, xanthan gum, guar gum, locust bean gum, methyl cellulose, sodium alginate, tara gum, a pine-based hydrocolloid, a combination of hydrocolloid and/or hydrogel materials, and/or the like.

Step 206 of method 200 includes coating at least one side of the planar expanse of hydrocolloid-containing material with ozonated olive oil, such as the ozonated olive oil resulting from step 202. This step may be performed using a roll-to-roll coating machine such as is known in the art, or by any other suitable method. In any case, sufficient ozonated olive oil should be applied to the hydrocolloid-containing material to allow a desired degree of infusion and/or absorption of the ozonated olive oil into the hydrocolloid-containing material. As discussed previously, suitable ratios of ozonated olive oil to hydrocolloid material may be, for example, in the range of 0.0025 to 0.025 by weight; in the range of 0.005 to 0.010 by weight; in the range of 0.010 to 0.020 by weight; or in the range of 0.007 to 0.008 by weight.

Step 208 of method 200 includes applying a release layer to a side of the planar expanse of material. In some cases, this step may be performed before applying the ozonated olive oil to the hydrocolloid-containing material. In other cases, ozonated olive oil may be applied to the hydrocolloid-containing material before any release layer is applied. As discussed previously, suitable materials for a release layer used in step 208 may include, for example, polyethylene terephthalate (PET), polyurethane, and silicone, among others.

In still other embodiments of method 200, step 204 may include extruding the hydrocolloid-containing material onto a first or temporary release layer, step 206 may include applying ozonated olive oil to a side of the hydrocolloid material oppose the first release layer, and step 208 may include the application of a second, more permanent release layer. In such cases, the first release layer may or may not be removed before application of the second release layer. If the first release layer is removed before application of the second release layer, the second release layer may be applied to the same side of the hydrocolloid material as the first release layer, i.e., opposite the side coated with ozonated olive oil. If the first release layer is not removed before application of the second release layer, the second release layer may be applied to the opposite side of the hydrocolloid material from the first release layer, i.e., overlying the side coated with ozonated olive oil.

Step 210 of method 200 includes applying an optional outer sealing layer overlying the side of the planar expanse of material opposite the release layer. As discussed above, the outer sealing layer may be constructed from any material suitable for preventing or inhibiting penetration of bacteria, dirt, water, and/or other substances desired to be excluded from the underlying layers. Suitable outer layer materials include, for example, a non-woven fabric, a foam, a polyurethane film, and/or a combination of those or similar materials. In some cases, an outer side of the hydrocolloid material (i.e., the side furthest from a user's skin) may function at least in part as a protective layer, and no separate outer sealing layer may be provided.

As with the release layer, the outer sealing layer may be applied (when provided), for example, in a roll-to-roll manufacturing process (also known as a “reel-to-reel” manufacturing process), wherein the planar expanse of material, including the hydrocolloid-containing material infused with ozonated olive oil and possibly also a release layer, are brought together with a planar expanse of outer sealing layer material and run through a compression roller or simply adhered together without pressure.

In some cases, step 210 may be performed before application of the more permanent release layer as described in step 208, in which case step 208 may be performed after step 210. In any case, the result of method 200 including steps 202-210 (regardless of the order in which those steps are performed, or if any optional steps are omitted) is a planar expanse of material including a release layer on one side, an outer sealing layer on the other side, and hydrocolloid-containing material infused with ozonated olive oil sandwiched between the release layer and the outer sealing layer. As discussed above, in some cases the outer sealing layer may be an outer side of the hydrocolloid material, rather than a separate layer of material.

Step 212 of method 200 includes cutting the planar expanse of material resulting from the combination of all the previously performed steps into patches of one or more sizes suitable for application to human skin. For example, the material may be cut into circular patches of any desired diameter(s), such as 8 mm, 10 mm, 12 mm, 14 mm, and so forth. Alternatively or in addition, the material may be cut into shapes specifically configured to fit onto particular parts of a user's skin, particularly the user's facial skin, such as the nose, chin, cheek, brow, or the like.

In some cases, step 212 is performed before applying the final release layer, in which case the cut material may have the final release layer applied, and the patches may be packaged and sold with the cut shapes disposed on release layer sheets. In other cases, the cutting of step 212 is performed after applying the final release layer, in which case the cut shapes may be packaged and sold with the release layer cut into the same shapes. In some case, the release layer may be perforated to allow convenient separation of patches or subsets of patches.

### C. Illustrative Combinations and Additional Examples

This section describes additional aspects and features of a hydrocolloid-based skin patch, presented without limitation as a series of paragraphs, some or all of which may be alphanumerically designated for clarity and efficiency. Each of these paragraphs can be combined with one or more other paragraphs, and/or with disclosure from elsewhere in this application, in any suitable manner. Some of the paragraphs below expressly refer to and further limit other paragraphs, providing without limitation examples of some of the suitable combinations.

A. A skin patch for cosmetically treating acne, comprising:
an inner layer including a hydrocolloid material;
ozonated olive oil absorbed within the hydrocolloid material of the inner layer; and
a release layer overlying one side of the inner layer.

A1. The skin patch of paragraph A, further comprising an outer sealing layer overlying a side of the inner layer opposite the release layer.

A2. The skin patch of paragraph A, wherein the ratio of ozonated olive oil to hydrocolloid material in the inner layer is in the range of 0.0025 to 0.025 by weight.

A3. The skin patch of paragraph A2, wherein the ratio of ozonated olive oil to hydrocolloid material in the inner layer is in the range of 0.005 to 0.010 by weight.

A4. The skin patch of paragraph A3, wherein the ratio of ozonated olive oil to hydrocolloid material in the inner layer is in the range of 0.007 to 0.008 by weight.

A5. The skin patch of paragraph A, wherein the hydrocolloid material consists essentially of a pine-based hydrocolloid.

A6. The skin patch of paragraph A, wherein the ozonated olive oil is fully saturated with ozone.

A7. The skin patch of paragraph A, wherein the ozonated olive oil has a density in the range of 0.930 g/cm³ to 1.10 g/cm³.

A8. The skin patch of paragraph A7, wherein the ozonated olive oil has a density in the range of 0.980 g/cm³ to 0.995 g/cm³.

A9. The skin patch of paragraph A1, wherein the outer sealing layer is an outer side of the inner layer.

B. A method of manufacturing a skin patch for cosmetic acne treatment, comprising:
ozonating olive oil until the olive oil is fully saturated with ozone;
providing a planar expanse of material including a hydrocolloid material;
coating a side of the planar expanse of material with the ozonated olive oil;
applying a release layer to a side of the planar expanse of material; and
cutting the planar expanse of material of into patches of one or more sizes suitable for application to human skin.

B1. The method of paragraph B, wherein coating a side of the planar expanse of material with the ozonated olive oil includes coating exactly one side of the planar expanse of material with the ozonated olive oil.

B2. The method of paragraph B1, wherein the release layer is applied to the side of the planar expanse of material opposite the side of the planar expanse of material coated with ozonated olive oil.

B3. The method of paragraph B, wherein the ratio of ozonated olive oil to hydrocolloid material is in the range of 0.0025 to 0.025 by weight.

B4. The method of paragraph B3, wherein the ratio of ozonated olive oil to hydrocolloid material is in the range of 0.005 to 0.010 by weight.

B5. The method of paragraph B4, wherein the ratio of ozonated olive oil to hydrocolloid material is in the range of 0.007 to 0.008 by weight.

B6. The method of paragraph B, wherein the hydrocolloid material consists essentially of a pine-based hydrocolloid.

B7. The method of paragraph B, wherein the ozonated olive oil has a density in the range of 0.930 g/cm³ to 1.10 g/cm³.

B8. The method of paragraph B7, wherein the ozonated olive oil has a density in the range of 0.980 g/cm³ to 0.995 g/cm³.

B9. The method of paragraph B, further comprising an outer sealing layer to a side of the planar expanse of material opposite the release layer.

C. A skin patch for cosmetically treating acne, comprising:
an inner layer including a hydrocolloid;
ozonated olive oil absorbed within the hydrocolloid, wherein the density of the ozonated olive oil is in the range of 0.985 g/cm³ to 0.990 g/cm³, and the ratio of ozonated olive oil to hydrocolloid is in the range of 0.005 to 0.020 by weight; and
a release layer overlying one side of the inner layer.

C1. The skin patch of paragraph C, wherein the hydrocolloid consists essentially of a pine-based hydrocolloid, and the ratio of ozonated olive oil to hydrocolloid is in the range of 0.0070 to 0.015 by weight.

### Advantages, Features, and Benefits

The different embodiments and examples of the skin patches described herein provide several advantages over known solutions for cosmetically treating acne. For example, illustrative embodiments and examples described herein allow cosmetic treatment of acne that includes the benefits of both hydrocolloid material and ozonated olive oil.

Additionally, and among other benefits, illustrative embodiments and examples described herein allow cosmetic acne treatment using ozonated olive oil in the form of an adhesive patch.

Additionally, and among other benefits, illustrative embodiments and examples described herein provide improved cosmetic acne treatment and healing relative to treatments that employ only hydrocolloid or hydrogel materials.

No known system or device can perform these functions. However, not all embodiments and examples described herein provide the same advantages or the same degree of advantage.

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

1. A hydrocolloid-based skin patch, comprising:
an inner layer including a hydrocolloid material;
ozonated olive oil absorbed within the hydrocolloid material of the inner layer; and
a release layer overlying one side of the inner layer.

2. The skin patch of claim 1, further comprising an outer sealing layer overlying a side of the inner layer opposite the release layer.

3. The skin patch of claim 1, wherein a ratio of ozonated olive oil to hydrocolloid material in the inner layer is in a range of 0.0025 to 0.025 by weight.

4. The skin patch of claim 3, wherein the ratio of ozonated olive oil to hydrocolloid material in the inner layer is in the range of 0.005 to 0.010 by weight.

5. The skin patch of claim 4, wherein the ratio of ozonated olive oil to hydrocolloid material in the inner layer is in the range of 0.010 to 0.020 by weight.

6. The skin patch of claim 1, wherein the hydrocolloid material consists essentially of a pine-based hydrocolloid from a pine tree or a pine shrub.

7. The skin patch of claim 1, wherein the ozonated olive oil is fully saturated with ozone.

8. A hydrocolloid-based skin patch, comprising:
an inner layer including a hydrocolloid material;
ozonated olive oil absorbed within the hydrocolloid material of the inner layer; and
a release layer overlying one side of the inner layer,
wherein the ozonated olive oil is saturated with ozone and the ozonated olive oil has a density in a range of 0.930 g/cm³ to 1.10 g/cm³.

9. The skin patch of claim 8, wherein the ozonated olive oil has a density in a range of 0.980 g/cm³ to 0.995 g/cm³.

10-18. (canceled)

19. A skin patch for cosmetically treating acne, comprising:
an inner layer including a hydrocolloid;
a coating of ozonated olive oil absorbed within the hydrocolloid, wherein a density of the ozonated olive oil is in a range of 0.985 g/cm³ to 0.990 g/cm³, and a ratio of ozonated olive oil to hydrocolloid is in a range of 0.007 to 0.008 by weight; and
a release layer overlying one side of the inner layer.

20. The skin patch of claim 19, wherein the hydrocolloid consists essentially of a pine-based hydrocolloid from a pine tree or a pine shrub, and the ratio of ozonated olive oil to hydrocolloid is in the range of 0.0070 to 0.0075 by weight.
